SEQUENCE LISTING (1 sequence)

SEQ ID NO: 1 - protein, 266 aa, Serratia marcescens
MRFNNKMLALAALLFAAQASADTLESIDNCAVGCPTGGSSNVSIVRHAYTLNNNSTTKFANWVAYHITKDTPASGKTRNWKTDPALNPADTLAPADYTGANAALKVDRGHQAPLASLAGVSDWESLNYLSNITPQKSDLNQGAWARLEDQERKLIDRADISSVYTVTGPLYERDMGKLPGTQKAHTIPSAYWKVIFINNSPAVNHYAAFLFDQNTPKGADFCQFRVTVDEIEKRTGLIIWAGLPDDVQASLKSKPGVLPELMGCKN

(19) United States
(12) Patent Application Publication
(10) Pub. No.: US 2024/0409894 A1
(43) Pub. Date: Dec. 12, 2024

(54) METHOD FOR PRODUCING HIGH-DENSITY CULTURE CELLS

(71) Applicant: SAFI BIOTHERAPEUTICS INC., Wilmington, DE (US)

(72) Inventors: Guillaume Rousseau, Paris (FR); Marie-catherine Giarratana, Saint-Ouen (FR); Florent Mathieu, Clermont-Ferrand (FR); Fanny Rasschaert, Paris (FR)

(21) Appl. No.: 18/690,385

(22) PCT Filed: Sep. 8, 2022
(86) PCT No.: PCT/EP2022/075022

§ 371 Date: Mar. 8, 2024

(30) Foreign Application Priority Data
Sep. 8, 2021 (FR) FR2109408

(51) Int. Cl. C12N 5/078

(52) U.S. Cl. CPC C12N 5/0641; C12N 2501/73; C12N 2521/00

(57) ABSTRACT

The present invention relates to a method for producing cultured cells, comprising a step of culturing cells to be cultured in a perfusion bioreactor containing a culture medium and in which the culture medium is filtered at a bioreactor outlet by a filter, wherein the culture medium comprises at least one nuclease.

### FIELD OF THE INVENTION

The present invention relates to a method for producing cultured cells.

### TECHNICAL BACKGROUND

Batch and fed-batch cell cultures are the most widely used cell culture methods, both for recombinant protein production and for cultivated cell production. However, these methods are limited in terms of production volumes and yields.

In this context, cultivation using perfusion bioreactors appears to be an interesting alternative. However, a number of hurdles still need to be overcome before this type of cultivation can be used more frequently.

Perfusion bioreactors, especially when equipped with a tangential-flow filtration (TFF) system, are associated with significant cell lysis.

However, it has been shown that this cell lysis could be mitigated with an alternating tangential flow (ATF) system or by replacing the peristaltic pump classically used to ensure the flow of culture medium through the filter with a low-shear pump, such as a centrifugal pump (Wang et al. (2017) J. Biotechnol. 246:52-60).

Yet, these solutions are still associated with significant cell lysis. Moreover, they are limiting for very high-density cell culture.

It is therefore still necessary to find solutions to overcome the problem of cell lysis, particularly for high-density cell cultures.

### SUMMARY OF THE INVENTION

The present invention stems from the inventors' unexpected finding that adding a nuclease to a perfusion bioreactor reduced lysis of cultured cells.

The present invention thus relates to a method for producing cultured cells, comprising a step of culturing cells to be cultured in a perfusion bioreactor containing a culture medium and in which the culture medium is filtered at a bioreactor outlet by a filter, wherein the culture medium comprises at least one nuclease.

Advantageously, and unexpectedly, when applied to the production of cultured red blood cells, the method for producing cultured cells of the invention improves the terminal enucleation rate of the culture.

### DESCRIPTION OF THE INVENTION

As a preliminary point, it will be recalled that the term “comprising” means “including”, “containing” or “encompassing”, i.e. when an object “comprises” an element or several elements, elements other than those mentioned may also be included in the object. In contrast, the expression “consisting of” means “constituted by”, i.e. when an object “consists of” an element or several elements, the object cannot include elements other than those mentioned.

### Cells

Cells according to the invention are of any type.

Preferably, they are eukaryotic cells, more preferably animal cells, in particular bird or mammalian cells, more particularly human cells.

The cells to be cultured according to the invention can be primary culture cells or immortalized cell line cells.

Preferably, the cultured cells produced according to the method of the invention are cells of the erythroid lineage, cultured red blood cells or cultured meat cells.

As we understand it here, “cultured meat” is synonymous with “synthetic meat” or “clean meat”.

The cells to be cultured according to the invention may be stem cells, progenitors, or cells of an immortalized cell line of the erythroid lineage.

The stem cells may be embryonic stem cells (ESC), induced pluripotent stem cells (iPSC), or hematopoietic stem cells and/or progenitors (HSC/HP). Preferably, the method according to the invention uses hematopoietic stem cells (HSC) as the cell source.

Cells of an immortalized cell line of the erythroid lineage can be immortalized at the stage of an erythroid progenitor or an erythroid precursor, particularly at an early stage. Hematopoietic stem cells (HSC) can also be immortalized.

Immortalization is preferably carried out conditionally. These immortalized cells can then be passed indefinitely in vitro, cryopreserved and recovered, and conditionally produce fully differentiated red blood cells from a defined and well-characterized source. Conditional immortalization can be achieved by any process well known to those skilled in the art.

Embryonic stem cells (ESC) and induced pluripotent stem cells (iPSC) are pluripotent stem cells. These cells are capable of both differentiation into numerous cell types and of self-replication. They can maintain this pluripotent differentiation while multiplying by division. Embryonic stem cells refer to pluripotent stem cells derived from embryos at the blastocyst stage, the early stage of animal development. Induced pluripotent stem cells (iPSCs) are produced by introducing several types of transcription factor genes into somatic cells such as fibroblasts.

Embryonic stem cells (ESC) according to the invention are obtained by any means not requiring the destruction of human embryos. For example, using the technology described by Chung et al (Chung et al, Human Embryonic Stem Cell lines generated without embryo destruction, Cell Stem Cell (2008)). Furthermore, the method according to the invention does not use human embryos and is not intended to induce the development process of a human being.

According to an embodiment of the invention, said stem cells used in the method according to the invention are not human embryonic stem cells (hESC) and/or iPSCs.

The hematopoietic stem cells (HSC) used in the method according to the invention are multipotent cells. They are capable of differentiating into all blood cell differentiation lineages, and of self-replicating while maintaining their multipotency.

Cells of an immortalized erythroid lineage are cells already committed to the erythroid lineage but capable of self-replicating and under external control of differentiating into erythroid lineage cells.

The cells to be cultured according to the invention, in particular the hematopoietic stem cells and/or progenitors (HSC/HP) used in the method according to the invention, can come from any source, including umbilical/placental cord blood, peripheral blood, bone marrow, or apheresis collection, with or without prior mobilization.

The origin of stem cells and cells of an immortalized cell lineage of the erythroid lineage is not particularly limited as long as it is derived from a mammal. Preferred examples include humans, dogs, cats, mice, rats, rabbits, pigs, cows, horses, sheep, goats and the like, with humans being most preferred.

The cells used in the method according to the invention can produce, without limitation, red blood cells from universal donors, red blood cells from a rare blood group, red blood cells for personalized medicine (for example, autologous transfusion, possibly with genetic engineering) and red blood cells designed to include one or more proteins of interest.

In certain embodiments which may be combined with any of the preceding embodiments, said cells used in the method according to the invention may be isolated from a patient having a rare blood group comprising, without limitation, Oh, CDE/CDE, CdE/CdE, CwD−/CwD−, −D−/−D−, Rhnull, Rh: −51, LW (a−b+), LW (ab−), SsU−, SsU (+), pp, Pk, Lu (a+b−), Lu (ab−), Kp (a+b−), Kp (ab−), Js (a+b−), Ko, K: −11, Fy (ab−), Jk (ab−), Di (b−), I−, Yt (a−), Sc: −1, Co (a-), Co (ab−), Do (a−), Vel−, Ge−, Lan−, Lan (+), Gy (a−), Hy−, At (a−), Jr (a−), In (b−), Tc (a−), Cr (a−), Er (a−), Ok (a−), JMH− and En (a−).

According to an embodiment of the invention, said cells can be embryonic stem cells (ESC), preferably human (hESC) and preferably selected from the group consisting of lines H1, H9, HUES-1, HUES-2, HUES-3, HUES-7, CLO1 and pluripotent stem cells (iPSC), preferably human (hiPSC).

Preferably, said cells are hematopoietic stem cells (HSC), most preferably human.

In the case of cells derived from umbilical cord/placental blood or peripheral blood, bone marrow or apheresis, a specific CD34+ cell selection step can be carried out prior to step a) of the method according to the invention.

Apheresis is a technique for removing certain blood components using extracorporeal blood circulation. The components to be removed are separated by centrifugation and extracted, while those not removed are reinjected into the (blood) donor or the patient (therapeutic apheresis).

CD34+ (positive) means that the CD (differentiation cluster) 34 antigen is expressed on the cell surface. This antigen is a marker of hematopoietic stem cells and hematopoietic progenitor cells, and disappears as they differentiate. Similar cell populations also include CD133-positive cells.

If the cells of origin are ESCs, iPSCs or cells of an immortalized cell lineage of the erythroid lineage, pre-culture steps can be added upstream of the culture step in the bioreactor to multiply the cells and possibly commit them to a differentiation pathway, notably of the erythroid lineage.

Preferably, the cultured cells are cultured red blood cells and the cells to be cultured are erythroid stem or progenitor cells or cells of an immortalized cell line of the erythroid lineage.

Whatever the cell source, a preliminary freezing step of the cells to be cultured is often required for transport and preservation reasons. Cell freezing methods are well known to the state of the art, and include programmed temperature descent and the use of cryoprotectants such as lactose or dimethylsulfoxide (DMSO). When added to the medium, DMSO prevents the formation of intracellular and extracellular crystals in the cells during the freezing process.

Thus, in a particular embodiment of the invention, the method according to the invention comprises a cell thawing step, prior to the culture step in a perfusion bioreactor, in the case where the cells to be cultured are frozen. Cell thawing methods are well known to those skilled in the art.

Thawing is an important step in the process, particularly when DMSO has been used for freezing. This compound is cryopreservative as long as the cell suspension is kept in liquid nitrogen or nitrogen vapor. However, it becomes cytotoxic as soon as the cell suspension is thawed. DMSO therefore needs to be removed very quickly by several washing steps as soon as the cells are thawed, as is well known to those skilled in the art.

In other cases, the cells to be cultured may be fresh, i.e. the time between collection and culture is short enough not to require freezing, preferably less than 48 hours. This may be the case, for example, when the sampling center is located on the same site or close to the production center.

### Method of Culture

Perfusion is a continuous culture method in which cells are retained in the bioreactor or circulated and returned to the bioreactor, while used culture medium is evacuated, compensated for by the addition of perfusion fluid to renew the culture medium. The used and discharged culture medium therefore contains no cells. In the present case, the culture medium is filtered at a bioreactor outlet to yield a permeate.

The aim of the culture stage in a perfusion bioreactor according to the invention is to multiply the cultured cells and, in the case of the production of cultured red blood cells, to complete their differentiation to a reticulocyte stage, an enucleated cell corresponding to a young or immature red blood cell, or to a mature red blood cell stage.

The culture is carried out in a bioreactor suitable for perfusion culture. Numerous bioreactor models suitable for perfusion cell culture are known to the person skilled in the art.

The bioreactor preferably has a capacity of 0.5 to 5000 L. Preferably, the bioreactor has a capacity of at least 0.5, 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000 or 4000 L. Preferably, the bioreactor has a capacity of at most 5000, 4000, 3000, 2000, 1000, 900, 800, 700, 600, 500, 400, 300, 200, 100, 90, 80, 70, 60, 50, 40, 30, 20, 10, 9, 8, 7, 6, 5, 4, 3, 2 or 1 L.

Preferably, the bioreactor comprises gas exchange means to satisfy the oxygen requirements of the cells and to control the pH by controlling the supply and/or removal of carbon dioxide (CO₂). Preferably, the gas exchange means is low shear.

Preferably, at least one of the following growing conditions, more preferably all of them, are controlled or regulated:
Agitation;
pH;
Dissolved oxygen (DO);
Temperature;
Volume or level of the bioreactor;
Infusion rate;
Nutrient input, in particular carbohydrates, amino acids, vitamins and iron;
Input in growth factors, cytokines and/or hormones;
Fouling of the bioreactor and clogging of filter elements.

Preferably, the culture is carried out for a period of time sufficient to obtain a cell concentration greater than 30 million cells/ml. Preferably this period of time is from 5 days to 25 days, more preferably from 10 days to 20 days.

Preferably, the culture temperature is between 33° C. and 40° C., more preferably between 35° C. and 39° C., and even more preferably between 36° C. and 38° C.

Preferably, the culture pH is between 7 and 8, more preferably between 7.2 and 7.7.

Preferably, the culture OD is between 1% and 100%, more preferably between 10% and 100%.

Advantageously, the perfusion bioreactor culture step enables cultured cells to be concentrated to levels unattainable in batch and fed-batch culture, i.e. above 30 million cells/ml and up to 200 million cells/ml. Advantageously, the perfusion bioreactor culture step of the method of the invention can also be used to differentiate the cultured cells. Advantageously, in the case of the production of cultured red blood cells, the rate of enucleated cells at the end of the perfusion bioreactor culture stage exceeds 50%, 60%, 70% or 80%.

In an embodiment of the invention, the perfusion culture step is preceded by at least one batch or fed-batch bioreactor culture step.

In batch cultures, the medium is not renewed, so the cells have only a limited supply of nutrients. Fed-batch culture, on the other hand, corresponds to a batch culture with a supply of nutrients and/or culture medium.

The advantage of the batch or fed-batch bioreactor culture step(s) is that the cells to be cultured are pre-amplified and, in the case of the production of cultured red blood cells, the starting cells are engaged or differentiated, or their engagement or differentiation is enhanced, towards the erythroid lineage.

Thus, in the case of the production of cultured red blood cells, it is possible, in an embodiment of the invention, to continue the culture step in a batch or fed-batch bioreactor until the cultured cells are committed to the erythroid lineage. According to this embodiment of the invention, cells are considered to be sufficiently committed to the erythroid lineage when they display one or more characteristics specific to the erythroid lineage, such as a percentage of cells displaying the CD235 marker, measurable for example by flow cytometry, greater than 50%, or a percentage of cells with an erythroid phenotype, measurable for example by cytological counting after staining with the May-Grünwald Giemsa dye, greater than 50%.

One or more successive or iterative cultures in a batch or fed-batch bioreactor can be carried out, for example between 1 and 4 times.

The batch or fed-batch bioreactor model is not particularly limited as long as it can generally culture animal cells. Preferably, the bioreactor of step a) has a capacity of 0.5 to 5000 L, more preferably 0.5 to 500 L.

In an embodiment of the invention, the method for producing cultured cells according to the invention comprises a step for purifying the cultured cells obtained after the perfusion bioreactor culture step.

The purpose of the purification step is:
to wash cells to remove potentially toxic residues from the method; and
in the case of production of cultured red blood cells, to sort the cells in order to concentrate enucleated cells as much as possible.

The purification step may comprise one or more operations, in particular a particle sorting operation and a washing operation. The washing operation may be performed either before and/or after the particle sorting operation.

In the case of cultured red blood cell production, particle sorting increases the enucleated cell rate, in particular by eliminating erythroblasts and any residual myeloid cells. Erythroblasts are cultured cells that have not reached the stage of enucleated cell differentiation, i.e. into reticulocytes or red blood cells. Particle sorting also eliminates cellular waste, such as cell debris, DNA and pyrenocytes.

Particle sorting according to the invention can comprise at least one operation selected from the group consisting of tangential-flow filtration, dead-end filtration and elutriation.

Tangential-flow filtration is well known to those skilled in the art. It is a filtration process that separates particles from a liquid according to their size. In tangential-flow filtration, the flow of liquid is parallel to the filter, in contrast to dead-end filtration, in which the liquid flow is perpendicular to the filter. It is the pressure of the fluid that allows it to pass through the filter. As a result, smaller particles pass through the filter, while larger ones continue their journey via the liquid flow.

Dead-end filtration is well known to those skilled in the art. Its principle consists in retaining the particles to be eliminated inside a porous network that forms the filter. Filtration is based on 4 mechanisms: (i) particle/wall adhesion forces, (ii) inter-particle adhesion forces, (iii) steric hindrance and (iv) the drag force of the fluid on the particles. Its effectiveness depends in particular on the material, pore size, type of fiber entanglement and the ratio of filtration surface area to the quantity of material to be filtered.

Elutriation is a technique for the separation and particle size analysis of particles of different sizes. Elutriation is based on Stokes' law. A fluid containing cells is sent into a chamber at a known speed, where the particles are subjected to a controlled centrifugal force. The particles remain in suspension when the two forces (fluid drag and centrifugal force) cancel each other out.

Preferably, the particle sorting operation according to the invention comprises a succession of dead-end filtrations and optionally of elutriation.

In particular, the purpose of the washing operation is to reduce the quantities of toxic compounds potentially present in the cell culture from step b) to below their toxicity threshold.

The washing operation may comprise one or more centrifugations and/or one or more elutriations.

Centrifugation is well known to those skilled in the art. It is a method for separating compounds in a mixture on the basis of their density difference and drag, by subjecting them to unidirectional centrifugal force and possibly to an opposing flow.

Preferably, the washing step according to the invention comprises a succession of elutriation operations.

The particle sorting, washing and formulation stages are carried out in a period of less than 72 hours, and more preferably less than 12 hours.

### Culture Medium

Preferably, the bioreactor is fed with a perfusion liquid, which may comprise a culture medium.

The person skilled in the art is able to select or prepare a suitable culture medium according to the invention. Examples of suitable culture media are those described in the international publication WO2011/101468A1 and in the article Giarratana et al. (2011) “Proof of principle for transfusion of in vitro-generated red blood cells”, Blood 118:5071-5079.

The culture medium generally comprises a basal culture medium for eukaryotic cells, such as DMEM, IMDM, RPMI 1640, MEM or DMEM/F12, which are well known to those skilled in the art and widely available commercially.

The culture medium or perfusion fluid may also include plasma, in particular in an amount of 0.5% to 6% (v/v).

Preferably, the culture medium or perfusion fluid also includes nutrients and growth factors, cytokines and/or hormones.

Thus, the person skilled in the art is able to adapt the culture medium and perfusion fluid by adding certain components or by modulating the quantities of certain components, including sodium, potassium, calcium, magnesium, phosphorus, chlorine, various amino acids, various nucleosides, various vitamins, various antioxidants, fatty acids, carbohydrates and analogues, fetal bovine serum, human plasma, human serum, horse serum, heparin, cholesterol, ethanolamine, sodium selenite, monothioglycerol, mercaptoethanol, bovine serum albumin, human serum albumin, sodium pyruvate, polyethylene glycol, poloxamers, surfactants, lipid droplets, antibiotics, agar, collagen, methylcellulose, various cytokines, various hormones, various growth factors, various small molecules, various extracellular matrices and various cell adhesion molecules.

Examples of cytokines included in the culture medium or perfusion fluid include interleukin-1 (IL-1), interleukin-2 (IL-2), interleukin-3 (IL-3), interleukin-4 (IL-4), interleukin-5 (IL-5), interleukin-6 (IL-6), interleukin-7 (IL-7), interleukin-8 (IL-8), interleukin-9 (IL-9), interleukin-10 (IL-10), interleukin-11 (IL-11), interleukin-12 (IL-12), interleukin-13 (IL-13), interleukin-14 (IL-14), interleukin-15 (IL-15), interleukin-18 (IL-18)), Interleukin-21 (IL-21), interferon-A (IFN-α), interferon-β (IFN-β), interferon-γ (IFN-γ), granulocyte colony-stimulating factor (G-CSF), monocyte colony-stimulating factor (M-CSF), granulocyte-macrophage colony-stimulating factor (GM-CSF), stem cell factor (SCF), flk2/flt3 ligand (FL), leukemia inhibitory factor (LIF), oncostatin M (OM), erythropoietin (EPO), thrombopoietin (TPO) However, it is not limited to these.

The various small molecules included in the culture medium or perfusion fluid may include aryl hydrocarbon receptor antagonists such as StemRegenin1 (SR1), hematopoietic stem cell self-renewal agonists such as UM171, and the like, but are not limited to these.

Growth factors included in the culture medium or perfusion fluid may include transforming growth factor-α (TGF-α), transforming growth factor-β (TGF-β), macrophage inflammatory protein-Iα (MIP-Iα), epidermal growth factor (EGF), fibroblast growth factor-1, 2, 3, 4, 5, 6, 7, 8 or 9 (FGF-1, 2, 3, 4, 5, 6, 7, 8, 9), nerve cell growth factor (NGF), vascular endothelial growth factor (VEGF), hepatocyte growth factor (HGF), leukemia inhibitory factor (LIF), nexin I protease, nexin Il protease, platelet-derived growth factor (PDGF), cholinergic differentiation factor (CDF), various chemokines, Notch ligands (such as Delta1), Wnt proteins, angiopoietin-like proteins 2, 3, 5 or 7 (Angpt 2, 3, 5, 7), insulin-like growth factors (GF), insulin-like growth factor binding protein (IGFBP), pleiotrophin, and the like, but not limited to.

Hormones in the culture medium or perfusion fluid may comprise hormones, in particular from the glucocorticoid family, such as dexamethasone or hydrocortisone, from the thyroid hormone family, such as T3 and T4, ACTH, alpha-MSH or insulin.

Preferably, particularly in the case of cultured red blood cell production, the bioreactor is fed, notably via the perfusion fluid, a source of ferric iron. More preferably, the ferric iron source is a complex of ferric iron and a chelating agent, in particular citrate.

Preferably, the culture medium comprises transferrin, particularly recombinant transferrin. Preferably, the transferrin concentration in the bioreactor is from 10 to 3,000 μg/ml, more preferably from 10 to 500 μg/ml.

### Filter

The filter according to the invention removes the used culture medium in the form of a permeate, while maintaining the cultivated cells in the bioreactor.

The “cut-off”, or filter pore size, is defined as the molar mass of the smallest compound in the filtered medium whose retention by the filter is 90%. The cut-off is generally specified for commercial filters.

Preferably, the cut-off according to the invention is less than 5 μm, 1.2 μm, 0.22 μm, 0.05 μm, 76 kDa, 70 kDa, 60 kDa, 50 kDa, 40 kDa, 30 kDa, 20 kDa, 15 kDa, 10 kDa, 9 kDa, 8 kDa, 7 kDa, 6 kDa, 5 kDa, 4 kDa, 3 kDa, 2 kDa or 1 kDa. Preferably, the cut-off according to the invention is greater than 1 kDa, 2 kDa, 3 kDa, 4 kDa, 5 kDa, 6 kDa, 7 kDa, 8 kDa, 9 kDa, 10 kDa, 15 kDa, 20 kDa, 30 kDa, 40 kDa, 50 kDa, 60 kDa, 70 kDa, 76 kDa, 0.05 μm, 0.22 μm, 1.2 μm or 5 μm. Preferably, the cut-off according to the invention is from 1 kDa to 50 kDa, more preferably from 1 kDa to 15 kDa.

Preferably, the filter is a tangential-flow filtration system.

Tangential-flow filtration (TFF) is well known to those skilled in the art. It is a filtration process that separates particles from a liquid according to their size. In tangential filtration, the liquid flow is parallel to the filter, in contrast to dead-end filtration, in which the liquid flow is perpendicular to the filter. It is the pressure of the liquid that allows it to pass through the filter. As a result, smaller particles pass through the filter, while larger ones continue their journey via the liquid flow.

Preferably, the filter is associated with at least one pump. The pump ensures the flow of culture medium through/in the filter. The pump may be a peristaltic pump, a lobe pump, a circumferential piston pump, a twin screw pump, a diaphragm pump, a membrane pump or a centrifugal pump. Preferably, the pump is a centrifugal pump.

Alternatively, alternating tangential flow filtration (ATF) can be used. In this case, the culture medium flows back and forth through/in the filter. In the case of alternating tangential flow filtration, the filter is preferably combined with a diaphragm pump.

Preferably, the filter consists of hollow fibers or a filter cassette.

### Nuclease

As understood herein, a nuclease is a hydrolase that cleaves the phosphodiester bonds of nucleic acid strands between two nucleotides. The nucleic acids hydrolyzed by the nuclease according to the invention can be deoxyribonucleic acid (DNA) and/or ribonucleic acid (RNA).

The nuclease according to the invention can be an exonuclease or an endonuclease. Preferably it is an endonuclease.

The nuclease according to the invention can be a protein or a ribonucleic acid, in particular a ribozyme.

Preferably, the nuclease is of bacterial origin, in particular it is a Serratia marcescens nuclease. Preferably, the nuclease is as described in the UniProtKB database under reference P13717. More preferably, the nuclease has the sequence of amino acids 22 to 266, 23 to 266 or 25 to 266 of SEQ ID NO: 1. Preferably, the nuclease is a recombinant protein, notably produced by Escherichia coli or Bacillus sp. By way of example, the nuclease according to the invention can be BENZONASE®, TURBONUCLEASE® or DENARASE®.

The nuclease can be supplied, in particular in one time or in several times or continuously, directly into the culture medium or bioreactor, or via the perfusion fluid.

Preferably, the nuclease is supplied in at least 1, 2, 3, 4, 5, 6, 7, 8, 9, 10, 15 or 20 times. Preferably, the nuclease is supplied in at most 20, 10, 9, 8, 7, 6, 5, 4, 3, 2 or 1 times. Preferably, the nuclease is supplied in 1 to 10 times, more preferably in 1 to 5 times.

Preferably, the nuclease is provided in a unit amount of at least 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1 or 2 U/mL. Preferably, the nuclease is provided in a unit amount of at most 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006 or 0.005 U/mL. Preferably, the nuclease is provided in a unit amount of from 0.005 to 1 U/mL, more preferably of from 0.05 to 0.5 U/ml. The expression “unit amount” refers to the amount of nuclease per addition.

Preferably, the nuclease is at a concentration in the bioreactor or in the culture medium, in particular averaged over the duration of the culture or at the end of the culture, of at least 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1 or 2 U/mL. Preferably, the nuclease is at a concentration, in the bioreactor or in the culture medium, in particular averaged over the duration of the culture or at the end of the culture, of at most 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006 or 0.005 U/mL. Preferably, the nuclease is at a concentration, in the bioreactor or in the culture medium, particularly averaged over the culture time or at the end of culture, of from 0.005 to 1 U/mL, more preferably of from 0.4 to 0.8 U/mL.

The invention will be further explained with the aid of the following non-limiting Example and Figures.

### DESCRIPTION OF FIGURES

FIG. 1 is a graph showing the LDH concentration corresponding to the level of cell lysis ([LDH], mU/mL) relative to the cell concentration ([C], cells/mL) at the end of culture (y-axis) as a function of the maximum cell concentration ([C]ₘₐₓ, cells/mL, x-axis) at the end of the production of cultured red blood cells according to the method of the invention in the presence (star symbol (*), dotted line) and absence (rhombus symbol), solid line) of nuclease in the culture medium.

FIG. 2 is a graph showing the percentage of negative Hoechst (−), corresponding to the percentage of enucleated cells (y-axis, %) at the end of culture as a function of the maximum cell concentration ([C] max, cells/mL, x-axis) at the end of production of cultured red blood cells according to the method of the invention in the presence (star symbol (*), dotted line) and absence (rhombus symbol (⋄), solid line) of nuclease in the culture medium.

### EXAMPLE

The production of cultured red blood cells was carried out with and without the addition of nuclease during the perfusion bioreactor culture stage of the method according to the invention.

Briefly, the cells cultured according to the invention are total nucleated cells collected by cytapheresis from volunteer donors previously mobilized with G-CSF.

A first step of the method according to the invention is carried out over 7 days (from D1 to D7) in fed-batch at a temperature of 37° C., under a 5% CO2 atmosphere and in a culture medium adapted from that described by Giarratana et al. (2011) “Proof of principle for transfusion of in vitro-generated red blood cells”, Blood 118:5071-5079 for the first step of the expansion procedure described in the article (page 5072). Halfway through this stage, fresh culture medium is added to the culture to dilute it by half (the same volume of culture medium is added as the volume initially present).

A second stage of the method according to the invention is carried out over 15 days (D8 to D22) in a 2 L perfusion bioreactor equipped with a tangential-flow filtration system and a centrifugal (TFF) or diaphragm (ATF) pump. Culture is carried out at 37° C., under a 5% CO2 atmosphere, with a culture medium similar to that of step a), except that IL-3 and glucocorticoid are absent. Punctual SCF and EPO additions are also made, as well as a continuous input of iron.

The second step is carried out in the absence or presence of a nuclease (Benzonase®, Merck) added three times to the culture medium at a final concentration of around 0.5 U/mL.

Several cultures are carried out, and the concentration of lactate dehydrogenase ([LDH]) in the culture medium is measured at the end of the cultures, as well as the concentration of cells ([C]) at the end of the culture and the maximum concentration of cells reached during the culture ([C]ₘₐₓ). The amount of LDH measured in the supernatant per cell produced is representative of cumulative cell lysis during culture. LDH concentration is measured using the Cedex Bio analyzer (Roche).

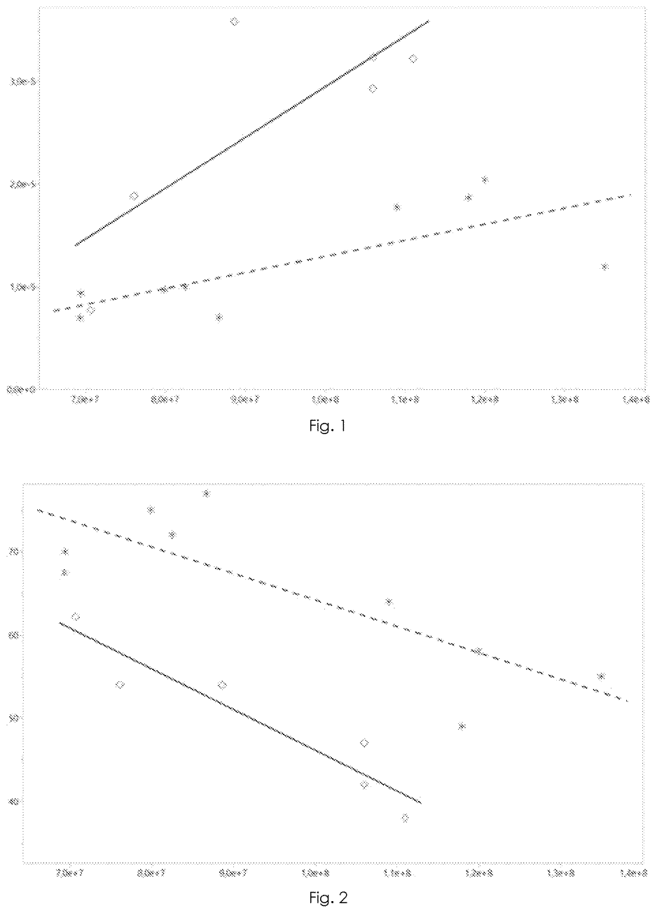

FIG. 1 shows that the addition of nuclease to the culture medium significantly reduces cell lysis.

In addition, several cultures are carried out and, at the end of the cultures, the percentage of enucleated cells is measured, i.e. cells measured as “negative” by flow cytometry following labelling with the Hoechst molecule (Hoechst 33258 solution, Sigma), as well as the maximum concentration of cells reached during the culture 5 [C]ₘₐₓ).

FIG. 2 shows that the addition of nuclease to the culture medium greatly increases the percentage of enucleated cells at the end of the culture.

1. A method for producing cultured cells, comprising a step of culturing cells to be cultured in a perfusion bioreactor containing a culture medium and in which the culture medium is filtered at a bioreactor outlet through a filter, wherein the culture medium comprises at least one nuclease.

2. The method for producing cultured cells according to claim 1, wherein the cells are eukaryotic cells.

3. The method for producing cultured cells according to claim 1, wherein the cultured cells are red blood cells and the cells to be cultured are stem cells or cells of an immortalized cell line of the erythroid lineage.

4. The method for producing cultured cells according to claim 3, wherein the cells to be cultured are embryonic stem cells (ESC), induced pluripotent stem cells (iPSC) or hematopoietic stem cells and/or progenitors (HSC/HP).

5. The method for producing cultured cells according to claim 3, wherein the cells to be cultured are cells of an immortalized cell line of the erythroid lineage.

6. The method for producing cultured cells according to claim 3, wherein the cells to be cultured are derived from umbilical/placental cord blood, peripheral blood, bone marrow, or apheresis collection.

7. The method for producing cultured cells according to claim 1, wherein the nuclease is an exonuclease or an endonuclease.

8. The method for producing cultured cells according to claim 1, wherein the nuclease is a recombinant protein.

9. The method for producing cultured cells according to claim 1, wherein the nuclease is of bacterial origin.

10. The method for producing cultured cells according to claim 1, wherein the nuclease is at a concentration of at least 0.005 U/ml.

11. The method for producing cultured cells according to claim 1, wherein the bioreactor filter is a tangential-flow filtration system.

12. The method for producing cultured cells according to claim 1, wherein the bioreactor filter consists of hollow fibers.

13. The method for producing cultured cells according to claim 1, wherein the filter has a cut-off below 76 kDa.

14. The method for producing cultured cells according to claim 1, wherein the cells are mammalian cells.

15. The method for producing cultured cells according to claim 5, wherein the cells to be cultured are erythroid progenitors or early erythroid precursors.

16. The method for producing cultured cells according to claim 7, wherein the nuclease is an endonuclease.

17. The method for producing cultured cells according to claim 9, wherein the nuclease is from Serratia marcescens.

18. The method for producing cultured cells according to claim 10, wherein the nuclease is at a concentration of at least 0.05 U/ml.

19. The method for producing cultured cells according to claim 10, wherein the nuclease is at a concentration of at least 0.5 U/ml.

20. The method for producing cultured cells according to claim 13, wherein the filter has a cut-off below 50 kDa, preferably below 15 kDa.
